(19) United States
(12) Patent Application Publication
(10) Pub. No.: US 2008/0108950 A1
(43) Pub. Date: May 8, 2008

(54) CORKSCREW HELICAL INSERTER PORT

(76) Inventors: Robert F. Rioux, Ashland, MA (US); David Danielsen, Westborough, MA (US); Christopher Pearson, North Grafton, MA (US)

Correspondence Address: FAY KAPLUN & MARCIN, LLP, 150 BROADWAY, SUITE 702, NEW YORK, NY, 10038, US

(21) Appl. No.: 11/834,186

(22) Filed: Aug. 6, 2007

Related U.S. Application Data
(60) Provisional application No. 60/856,978, filed on Nov. 3, 2006.

(51) Int. Cl. A61M 5/00

(52) U.S. Cl. 604/181

(57) ABSTRACT

Described is a method and a device for inserting a helical member into a living body. The device may include a handle having an actuator lever rotatably coupled thereto. The device may also include a helical member which has a tissue piercing distal tip. The helical member is coupled to the handle via a linkage operating so that, as the actuator lever is rotated in a first direction relative to the handle, the helical member is rotated and moved distally to screw into tissue along a substantially helical path.

### PRIORITY CLAIM

This invention claims priority to U.S. Provisional Patent Application Ser. No. 60/856,978 entitled “Corkscrew Helical Inserter Port” filed Nov. 3, 2006, the disclosure of which is incorporated, in its entirety, herein.

### BACKGROUND INFORMATION

Implantable infusion ports are routinely used to provide semi-permanent, repeated access to the vascular system to facilitate the provision fluids thereto and/or the withdrawal therefrom without requiring the repeated insertion of a needle into a blood vessel. Such infusion ports, which may be implanted subcutaneously or flush with the skin or which may be sutured to the skin, typically include a resilient self-sealing surface, or septum, serving as a barrier between the interior of the infusion port and the surrounding environment. Such a port is accessed by piercing the septum which is generally formed of silicone or another polymeric element that can withstand repeated piercing while continuing to reseal the puncture pore, or pathway, after the needle has been withdrawn. However, after multiple injections, the durability of the septum deteriorates, eventually reaching a point at which it is no longer be able to provide a dependable seal, requiring replacement of the septum and/or the infusion port, increasing discomfort and introducing risks such as infection and blood vessel damage.

### SUMMARY OF THE INVENTION

The present invention relates to a device for inserting a helical member into a living body. The device may include a handle which has an actuator lever rotatably coupled thereto. The device may also include a helical member which has a tissue piercing distal tip. The helical member is coupled to the handle via a linkage operating so that, as the actuator lever is rotated in a first direction relative to the handle, the helical member is rotated and moved distally to screw into tissue along a substantially helical path.

In another exemplary embodiment of the present invention The device may also include a fluid line which is coupled to a proximal end of the helical member. The helical member may include a lumen extending therethrough to an opening formed in the distal tip.

In another exemplary embodiment of the present invention, the device may also include a substantially straight needle coupled to the handle for movement with the helical member. The needle includes a lumen extending therethrough to an opening in a distal tip thereof.

In another exemplary embodiment of the present invention, the straight needle of the device extends substantially along a central axis of the helical member.

In another exemplary embodiment of the present invention, the straight needle of the device rotates with the helical member.

In another exemplary embodiment of the present invention, the straight needle of the device is non-rotatably coupled to the handle.

In another exemplary embodiment of the present invention, the device may include a rack member slidably coupled to the handle for movement relative thereto proximally and distally along an axis. The linkage includes a geared surface formed on the handle mating with a corresponding geared surface on the rack member.

In another exemplary embodiment of the present invention, the device may also include a helical member guide slidably receiving the helical member so that, to move proximally and distally therethrough. The helical member is forced to rotate about the axis.

In another exemplary embodiment of the present invention, the device may also include a mounting surface contoured and positioned to rest against a portion of a subject's anatomy to stabilize the device during use.

In another exemplary embodiment of the present invention, the device may also include a rotatable coupling fluidly coupling the lumen of the helical member to the fluid line.

In another exemplary embodiment of the present invention, a diameter of the helical member of the device is between 17 and 22 gauge.

In another exemplary embodiment of the present invention, a pitch of the helical member of the device is selected so that adjacent points are separated from one another as to allow 1-3 coils per inch.

In another exemplary embodiment of the present invention, a pitch of the helical member of the device is between 0.25 inches to 1 inch.

In another exemplary embodiment of the present invention, an inside coil diameter of the helical member of the device is between 0.125 inches to 0.50 inches.

The present invention also relates to a method for inserting a needle into a subcutaneous port. An injection device is aligned so that a helical member of the device is positioned over a portion of skin covering a subcutaneously implanted port. A lever of the injection device is rotated in a first direction relative to a handle coupled thereto to move a tissue piercing distal tip of the helical member distally toward the portion of skin while rotating the helical member so that the tissue piercing distal tip screws itself into the portion of skin and passes through the portion of skin and a resealable septum of the port along a helical path to enter a reservoir of the port. The lever is rotated in a direction opposite the first direction to withdraw the helical member from the septum and the skin along the helical path.

In another exemplary embodiment of the present invention, the helical member includes a fluid lumen extending therethrough to an opening formed in the tissue piercing distal tip. An injection of fluid to or a withdrawal of fluid from the port via the fluid lumen is performed.

In another exemplary embodiment of the present invention, the injection device includes a substantially straight needle extending substantially parallel to a central axis of the helical member. The needle includes a fluid lumen extending therethrough to an opening formed in the tissue piercing distal tip. An injection of fluid to or a withdrawal of fluid from the port via the fluid lumen is performed.

### BRIEF DESCRIPTION OF THE DRAWINGS

FIG. 1 is an exemplary injection assembly according to the present invention;

FIG. 2 shows a cross section of a septum showing a conventional needle puncture pathway;

FIG. 3 is a three-dimensional representation of the conventional needle puncture pathway of FIG. 2;

FIG. 4 shows a section of a septum showing a helical needle puncture pathway according to the present invention;

FIG. 5 shows a three-dimensional representation of the helical needle puncture pathway of FIG. 4;

FIG. 6 is a diagram showing an alternative embodiment of an injection assembly anchoring a straight needle;

FIG. 7 shows an acute needle tip for use in conjunction with the injection assembly according to the present invention;

FIG. 8 shows a flat needle tip for use in conjunction with the injection assembly according to the present invention; and

FIG. 9 shows a side view of a portion of a needle for use in conjunction with the injection assembly according to the invention.

### DETAILED DESCRIPTION

The present invention may be further understood with reference to the following description and the appended drawings, wherein like elements are referred to with the same reference numerals. The invention relates to methods and devices for needle delivery into an implantable port and more specifically relates to a helical injection assembly for use in conjunction with a self-sealing implantable infusion port. Throughout the specification, reference is made to surfaces of the septa and various planes relative to these septa. For example, reference may be made to an angle at which a needle passes through a septum relative to a plane of an outer surface of the septum. However, those skilled in the art will understand that this does not mean that the surface is planar. Rather, this simply refers to a plane most consistent with the orientation of the outer surface. For example, for a generally disc shaped septum, this refers to the angle of the needle relative to a plane symmetrically located with respect to inner and outer surfaces of the septum. Using this convention, the axial direction is defined as substantially perpendicular to this plane (i.e., the most direct path through the surface to a reservoir within the port) while the radial direction refers to lines substantially parallel to this plane.

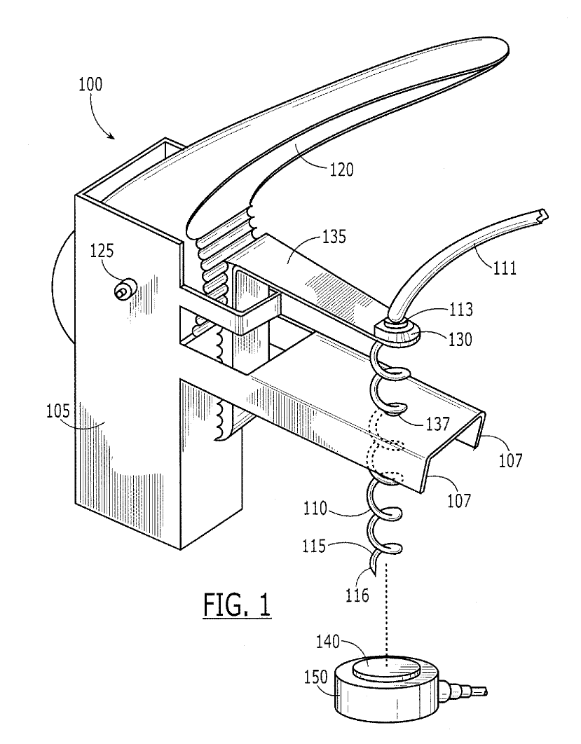

As shown in FIG. 1, an exemplary assembly 100 for injecting and/or withdrawing fluids via an infusion port 150 comprises a body 105, a helical needle 110 including a sharp distal needle tip 115, a plunger lever 120, a rotatable locknut 130, and a needle support 135. The tip 115 includes an aperture 116 which opens a lumen 117 extending through the needle 110 to the exterior. A proximal end of the needle 110 is coupled to an inlet/outlet tube 111 which extends proximally from the needle 110 to a source of fluid to be injected or to a reservoir or other structure for receiving fluids withdrawn from the body. The tube 111 is coupled to the needle 110 by a rotating coupling 113 which maintains fluid communication between the lumen 117 and a lumen of the tube 111 while allowing relative rotation between the needle 110 and the tube 111. According to this embodiment of the present invention, the tube 111 may connect with the rotating coupling 113 of the assembly 100 via a luer attachment. Once attached to the assembly 100, the tube and the luer attachment may freely rotate about the rotating coupling 113 while remaining in fluid communication with the lumen 117.

In another embodiment of the present application, the rotating coupling 113 may be a rotating fluid coupler having a bayonet design. Specifically, the bayonet design of the rotating coupler 113 may include male and female thermoplastic components with an o-ring between the male and female components allowing the coupling 113 to freely rotate while maintaining a fluid seal between the tube 111 and the lumen 117.

In a further alternative embodiment of the present invention, the tube 111 may be fixedly attached to the needle 110 in order to maintain fluid communication between the tube 111 and the lumen 117. According to this embodiment, the tube 111 may rotate with needle 110 during the insertion of the needle 110. As described below the pitch of the needle may be selected to control an amount of rotation of the needle 110 as it is inserted. For example, depending on its length, a needle 110 with a pitch of one to three coils per inch may complete only 2 to three rotations during insertion. In this case, a tube 111 connected to the needle 110 may simply be allowed to wind without substantially impacting the flow through the lumen 117.

A rotatable locknut 130 or other known coupling may be used to couple the proximal end of the helical needle 110 to the needle support 135 while allowing the needle 110 to freely rotate relative to the needle support 135 about an injection/withdrawal axis of the needle. The plunger lever 120 according to this embodiment includes a geared surface 122, teeth of which mate with teeth of a geared surface 124 formed on the needle support 135. The mating geared surfaces 122, 124 ensure that the needle support 135 is raised or lowered as the plunger lever 120 is rotated relative to the body 105 about a hinge pin 125. Rotation of the plunger lever 120 clockwise as seen in FIG. 1 moves the needle 110 in an injection direction while rotation of the plunger lever 120 counterclockwise moves the needle 110 in a withdrawal direction. The body 105 according to this embodiment of the invention includes optional anchor arms 107 distal to the needle support 135 for securely attaching the assembly 100 in a desired location on or near the body or to provide a stable base on which to rest the assembly 100 against the patient's body. The assembly 100 further includes an injection guide 136 including a guide aperture 137 formed therein with the needle 110 passing through the guide aperture 137. The guide aperture 137 is offset from the injection/withdrawal axis by a distance substantially equal to a radius of the helix of the needle 110 so that movement of the needle 110 proximally and distally through the guide aperture 137 forces the needle 110 to rotate within the guide aperture 137. In this embodiment, the injection guide 136 is mounted between the anchor arms 107. However, those skilled in the art will understand that the injection guide 136 may be mounted at any point along the length of the needle 110 proximal of a position of the tip 115 of the needle 110 when the needle 110 is fully withdrawn. That is, as the needle support 135 moves the needle 110 distally through the guide aperture 137, the needle 110 rotates counterclockwise as viewed from a proximal side of the injection guide 136 as each point on the helix of the needle 110 passes through the fixed location of the guide aperture 137. As the needle 110 is withdrawn proximally, the guide aperture 137 directs the needle 110 to rotate in the opposite direction.

In use, the anchor arms 107 are placed against a desired part of the body so that the tip 115 of the needle 110 is aligned with the septum 140 of an infusion port and the plunger lever 120 is rotated clockwise as seen in FIG. 1 to move the needle support 135 and the needle 110 distally. As described above, as the needle 110 moves distally through the needle guide aperture 137, the needle 110 is rotated about a longitudinal injection/withdrawal axis thereof in an injection direction (i.e., clockwise for a helical needle 110 wrapped clockwise about the injection/withdrawal axis from the proximal end to the distal tip 115). The rotating tip 115 of the needle 110 contacts the surface of the septum 140 (e.g., after passing through the skin), and draws itself distally into the septum 140, passing through the septum 140 along a helical path 401, as shown in FIG. 5. When a desired depth of injection has been reached (e.g., when the distal tip 115 has passed completely through the septum 140 into a fluid chamber of the port 150) fluids may be injected and or withdrawn through the aperture 116 in a manner similar to that used with conventional needles.

After the fluid injection/withdrawal has been completed, the plunger lever 120 is rotated counterclockwise as seen in FIG. 1 to rotate the needle 110 in the withdrawal direction, drawing the needle 110 and the tip 115 proximally, through the septum 140 along the path 401 in a direction opposite to that in which it was inserted until the tip 115 is completely withdrawn from the septum 140 and the skin. This provides a stabilized and controlled means of inserting the helical needle 110 into the port 150 and withdrawing the needle 110 therefrom. Furthermore, as described above, to enhance stability of the assembly 100 during injection, anchoring arms 107 of the assembly 100 may be secured to a fixed location with the helical needle 110 in a desired alignment with the infusion port 150. Once the needle 110 and the infusion port 150 have been properly aligned and the anchoring arms 107 are fixed in position, the user gradually lowers the plunger lever 120 causing the connected helical needle 110 to descend and rotate toward the infusion port 150 until the tip 115 penetrates a septum 140 of the port 150 to establish fluid communication between a reservoir of the port 150 and an interior lumen (not shown) of the needle 110.

After administering fluids to the infusion port 150 (or alternatively, removing fluids therefrom), the helical needle 110 is withdrawn by raising the plunger lever 120 to rotate the helical needle 110 in the opposite direction while drawing the needle 110 through the septum 140 and out of infusion port 150.

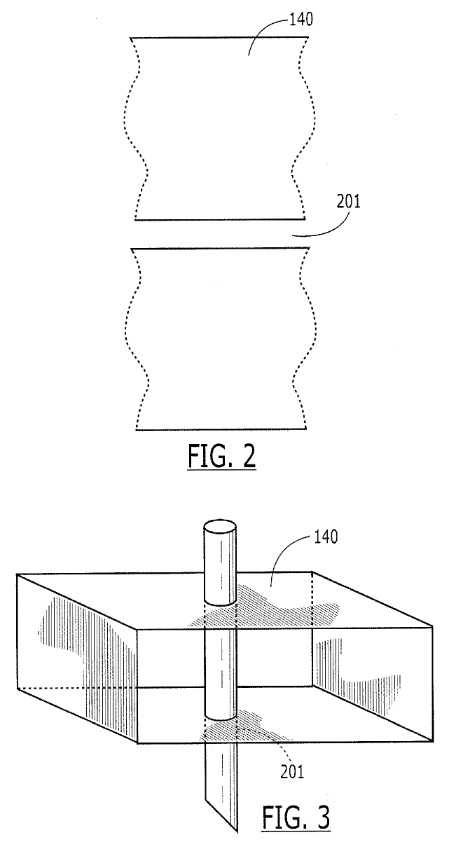

The helical puncture pathway along which the helical needle 110 penetrates the septum 140 enhances the self-sealing qualities and life of the septum 140. As shown in FIGS. 2 and 3, a puncture pathway 201 formed through a septum 140 by the insertion of a conventional needle (i.e., a Seldinger needle) is substantially linear and usually extends substantially perpendicular to a surface of the septum 140. This puncture displaces the resilient material of the septum 140 radially away from the puncture pathway 201 so that the resiliency of the septum 140 must push edges of this puncture pathway radially inward toward one another to reseal itself.

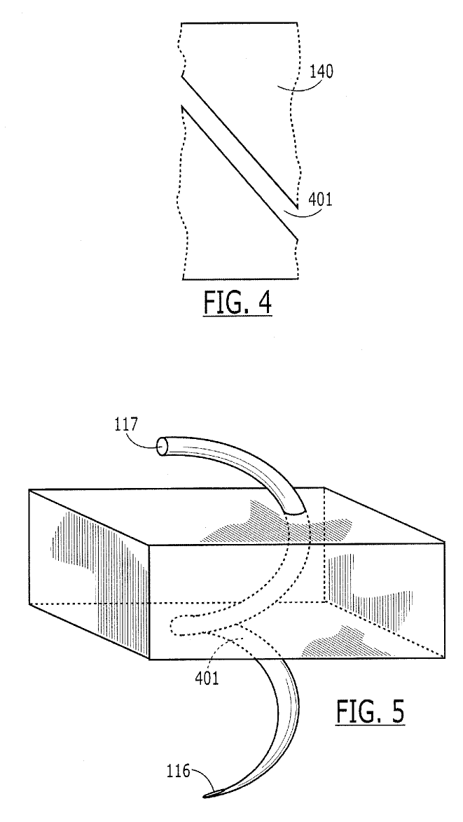

As shown in FIGS. 4 and 5, a puncture pathway 401 formed by a helical needle according to the present invention differs from the conventional puncture pathway 201 in two significant respects. First, each puncture pathway 401 made by a the helical needle 110 is oblique in relation to an outer surface of the septum 140. These diagonal puncture pathways 401 wrap around the injection/withdrawal axis which is generally perpendicular to the outer surface of the septum 140 enhancing the ability of the septum 140 to reseal itself by increasing the length and, consequently, the surface area of the portions of the septum 140 defining the pathway 401. Furthermore, as the pathways 401 extend through the septum 140 oblique with respect to the outer surface, expansion of the septum 140 after withdrawal of the needle 110 both radially and axially aids in resealing the pathway 401 as would be understood by those skilled in the art. Improving the ability to reseal increases the durability of the septum 140 and the time during which the infusion port 150 will reliably perform, reducing the frequency of procedures to replace or service the port 150 and/or the septum 140.

In addition to the improvement in the resealing of the septum 140, the oblique angle of the puncture path 401 creates a physical barrier enhancing the resistance of the needle 110 to accidental withdrawal from the septum 140 during injection. In contrast, conventional needles are retained within their linear needle pathways only by the friction applied to that portion of the needle extending through the septum. However, in addition to the mechanical resistance the septum provides to pulling out a helical needle, the friction force applied to the helical needle is longer as the helical path 401 is longer than that for conventional needles. That is, the downward rotation causes the helical needle 100 to spiral through the resilient material in a circular pattern having a diameter substantially the same as that of the helical needle 110, anchoring the needle 110 in the septum 140.

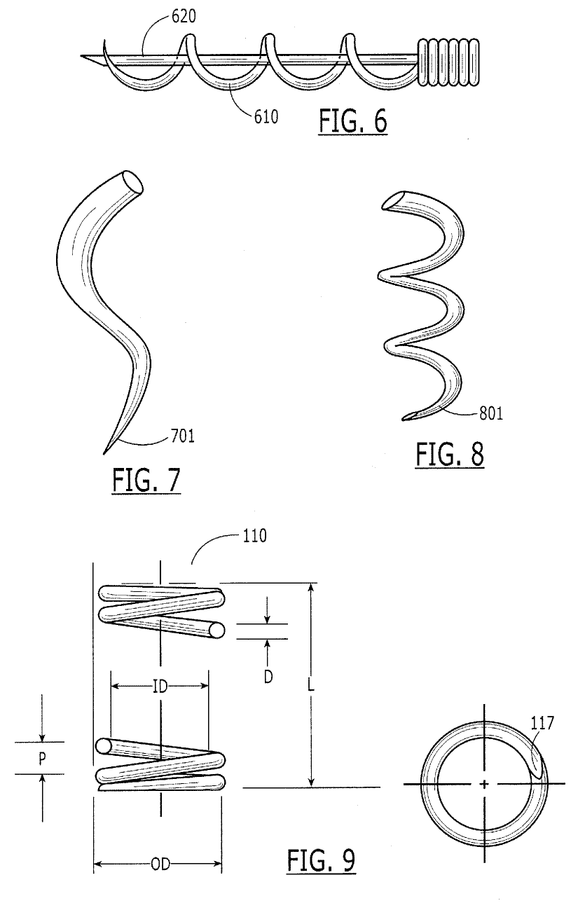

As shown in FIG. 6, a further exemplary embodiment of the present invention utilizes an assembly 100′ as an anchoring mechanism aiding in the insertion of a conventional straight needle 620 into an infusion port 150. As the helical anchor 610 of the assembly 100′ is not used for injections, it need not contain a lumen. The straight 620 needle is preferably coupled to the helical anchor 610 so that axial movement of the helical anchor 610 causes a corresponding axial movement of the straight needle 620. The straight needle 620 in this embodiment, is located within the coil formed by the helical anchor 620 and preferably extends substantially along a central axis of the coil.

The helical anchor 610 is preferably actuated by a mechanism substantially the same as that described above in regard to FIG. 1 with the helical anchor 610 and the needle 620 coupled to a rotatable locknut similar to the locknut 130 of the embodiment of FIG. 1. Those skilled in the art will understand that, alternatively, the needle 620 may be coupled to a non-rotatable component as the needle 620 need no rotate during injection. Raising and lowering the assembly 100′ is substantially the same as that described above for the embodiment of FIGS. 1-5 so that, as the helical anchor 610 is screwed into the septum 140, the needle 620 is drawn into the septum until a distal end of the needle 620 and an opening to an internal lumen thereof are within the port 150. When screwed into the septum 140, the helical anchor 610 provides a secure connection between the assembly 100′ and the infusion port 150. However, as stated above, these alternative systems and method may include the addition of the attachable straight needle 620.

Alternatively, the straight needle 620 may initially be decoupled from the assembly 100′. After the helical anchor 610 has been screwed into the septum 140, the needle 620 may then be inserted through an opening in the rotatable locknut 135 to pass along the central axis of the coil to penetrate the septum 140. In addition, the needle 620 may include a latch to secure the needle to the assembly 100′ in a desired position (e.g., at a depth at which the distal opening of the needle 620 is open to a reservoir of the port 150) and a coupling for attachment to a fluid supply/withdrawal line. As would be understood by those skilled in the art, the coupling for attachment to a fluid supply/withdrawal line may be a luer or other known coupling. Thus, the needle 620 will be anchored in the desired position by the assembly 100′ and may be withdrawn automatically as the helical anchor 610 is unscrewed from the septum 140.

In further alternate embodiments, the geometry of the needle tip 115 of the helical needle 110 may be modified to minimize damage. For example, as shown in FIGS. 7 and 8, the pitch of the coil of a helical needle of anchor may be varied to optimize a path of the tips 701, 801 through the septum 140 and/or to prevent damage thereto. Distal surfaces of the reservoirs of ports such as the port 150 are often formed of substantially rigid material to act as needle stops. Thus, as would be understood by those skilled in the art, the closer the tip of the helical needle/anchor is to parallel with such a distal surface of a reservoir, the less likely it will be that the tip will be damaged by contact therewith. Of course, those skilled in the art will understand that the pitch of the helical needle/anchor may preferably be selected to optimize its positive impact on the resealing of the septum 140 while minimizing the possibility of damage to the needle tip. It is important to note that a further alternate embodiment for the geometry of the needle 110 may be a hybrid between the needles illustrated in FIGS. 7 and 8. Accordingly, the alternative embodiment of the needle 110 may have loose coils as depicted in FIG. 7 with a deflected tip (e.g., extending substantially perpendicular to the injection/withdrawal axis) similar to the 801 of FIG. 8. As those skilled in the art will understand, the deflected tip minimizes damage to the needle tip 115 when the needle tip 115 makes contact with the base of the port 150.

FIG. 9 shows an embodiment of the helical needle 110, wherein the needle 110 has a wire diameter D, an inside coil diameter ID, an outside coil diameter OD, an overall length L and a pitch P (i.e., the distance between centers of adjacent coils of the needle 110). Those skilled in the art will understand that the pitch P of the needle 110 determines how tightly or loosely the coils of the needle 110 are positioned with a shorter pitch resulting in a tighter coil. Alternatively, the tightness of the coils may also be measured by specifying the number of active coils within a set length. For example, the tightness of the needle 110 may be described as a number of coils per inch. The needle 110 may be a loose coil (i.e., a low number of coils per inch) in order to minimize the number of revolutions required to penetrate the septum 140 of the port 150. In reference to FIG. 9, an exemplary helical needle 110 has a D value within the range of 17-22 gauge, a coil ID within the range of 0.125-0.5 inches and a coil OD in the range of 0.130-0.525 inches. Depending on the depth at which the port to be accessed has been implanted, the exemplary needle 110 preferably has a length L within the range of 0.25-1 inch and a pitch P of 1 to 3 coils per inch. Those skilled in the art will understand that the aforementioned ranges for the dimensions of the needle 110 are merely intended to serve as exemplary values and that any of these values may be expanded outside the described ranges to suit the particular needs of specific applications.

The present invention has been described with reference to specific embodiments. However, other embodiments may be devised that are applicable to other types of catheters and procedures. Accordingly, various modifications and changes may be made to the embodiments, without departing from the broadest spirit and scope of the present invention as set forth in the claims that follow. The specification and drawings are accordingly to be regarded in an illustrative rather than restrictive illustrative rather than restrictive sense.

What is claimed is:

1. A device for inserting a helical member into a living body, comprising:
a handle including an actuator lever rotatably coupled thereto; and
a helical member including a tissue piercing distal tip, the helical member being coupled to the handle via a linkage operating so that, as the actuator lever is rotated in a first direction relative to the handle, the helical member is rotated and moved distally to screw into tissue along a substantially helical path.

2. The device according to claim 1, further comprising:
a fluid line coupled to a proximal end of the helical member,
wherein the helical member includes a lumen extending therethrough to an opening formed in the distal tip.

3. The device according to claim 1, further comprising:
a substantially straight needle coupled to the handle for movement with the helical member, the needle including a lumen extending therethrough to an opening in a distal tip thereof.

4. The device according to claim 3, wherein the straight needle extends substantially along a central axis of the helical member.

5. The device according to claim 3, wherein the straight needle rotates with the helical member.

6. The device according to claim 3, wherein the straight needle is non-rotatably coupled to the handle.

7. The device according to claim 1, further comprising:
a rack member slidably coupled to the handle for movement relative thereto proximally and distally along an axis,
wherein the linkage includes a geared surface formed on the handle mating with a corresponding geared surface on the rack member.

8. The device according to claim 7, further comprising:
a helical member guide slidably receiving the helical member so that, to move proximally and distally therethrough, the helical member is forced to rotate about the axis.

9. The device according to claim 1, further comprising:
a mounting surface contoured and positioned to rest against a portion of a subject's anatomy to stabilize the device during use.

10. The device according to claim 2, further comprising:
a rotatable coupling fluidly coupling the lumen of the helical member to the fluid line.

11. The device according to claim 1, wherein a diameter of the helical member is between 17 and 22 gauge.

12. The device according claim 1, wherein a pitch of the helical member is selected so that adjacent points are separated from one another as to allow 1-3 coils per inch.

13. The device according to claim 1, wherein a pitch of the helical member is between 0.25 inches to 1 inch.

14. The device according to claim 1, wherein an inside coil diameter of the helical member is between 0.125 inches to 0.50 inches.

15. A method for inserting a needle into a subcutaneous port, comprising:
aligning an injection device so that a helical member of the device is positioned over a portion of skin covering a subcutaneously implanted port;
rotating a lever of the injection device in a first direction relative to a handle coupled thereto to move a tissue piercing distal tip of the helical member distally toward the portion of skin while rotating the helical member so that the tissue piercing distal tip screws itself into the portion of skin and passes through the portion of skin and a resealable septum of the port along a helical path to enter a reservoir of the port; and
rotating the lever in a direction opposite the first direction to withdraw the helical member from the septum and the skin along the helical path.

16. The method according to claim 15, wherein the helical member includes a fluid lumen extending therethrough to an opening formed in the tissue piercing distal tip, and the method further comprising the step of:
performing one of an injection of fluid to and a withdrawal of fluid from the port via the fluid lumen.

17. The method according to claim 15, wherein the injection device further includes a substantially straight needle extending substantially parallel to a central axis of the helical member, the needle including a fluid lumen extending therethrough to an opening formed in the tissue piercing distal tip, and wherein the method further comprising the step of:
performing one of an injection of fluid to and a withdrawal of fluid from the port via the fluid lumen.
